(19) United States
(12) Patent Application Publication
(10) Pub. No.: US 2004/0014043 A1
(43) Pub. Date: Jan. 22, 2004

(54) Sensitizer-labeled analyte detection

(75) Inventors: Derek W.K. Levison, Jackson, NJ (US); Uwe Moller, Berlin (DE); Stuart Levison, Jackson, NJ (US)

Correspondence Address: Daniel A. Scola, Jr., HOFFMANN & BARON, LLP, 6900 Jericho Turnpike, Syosset, NY, 11791, US

(73) Assignee: emp Biotech GmbH, Berlin (DE)

(21) Appl. No.: 10/197,288

(22) Filed: Jul. 16, 2002

(51) Int. Cl. C12Q 1/68; G01N 33/53

(52) U.S. Cl. 435/6; 435/7.1

(57) ABSTRACT

The invention provides methods for detecting an analyte in a sample including the steps of: (a) exciting a sensitizer label on an analyte; (b) permitting energy from the excited sensitizer label to be transferred to and excite an acceptor molecule, whereby the sensitizer label returns to an unexcited state; (c) reacting the excited acceptor molecule with a chemiluminescent precursor to form a chemiluminescent compound which emits light in response to an activation source; (d) exposing the chemiluminescent compound to the activating source to produce a detectable signal; (e) detecting the signal; and (f) correlating the signal with the presence or absence of the analyte. The chemiluminescent precursor is desirably an olefin capable of being converted to a 1,2-dioxetane. Target amplification techniques, such as PCR, may be used to directly label a target analyte with a sensitizer.

### FIELD OF THE INVENTION

The invention relates generally to chemiluminescent assays for the detection of an analyte in a sample to be inspected. More particularly, the invention relates to chemiluminescent assays which utilize a sensitizer as a label conjugated with an analyte, in which the sensitizer becomes electronically excited and transfers its excess energy to other compounds in association therewith so as to cause such other compounds to produce a detectable signal that can be monitored and/or quantitated.

### BACKGROUND OF RELATED TECHNOLOGY

Recently, a variety of non-isotopic labeling methods have been developed to replace radioactive labels in DNA probe-based assays. It is most common in such methods to use marker enzymes to detect nucleic acid probes using either colormetric, chemiluminescent, biolumininescent or fluorescent methods. Each of these methods have been used reliably for both hybridization of DNA in probe-based assays for nucleic acid detection, as well as solid-phase immunochemical assays wherein the target molecule is typically an antigen of interest.

Regardless of the type of non-isotopic detection method used, the labels are measured directly with fluorophores (without use of enzymes) or indirectly using enzyme amplification schemes. Wherein the label is detected directly without an enzymatic reaction, sensitivity is generally less.

Chemiluminescence detection relies on a chemical reaction that generates light. It is this method which is now widely used for both nucleic acid detection as well as solid-based immuno detection due to its high sensitivity and wide variety of analysis methods ranging from manual film reading to instrumentation for processing images. Typically, commercially available chemiluminescent detection methods have employed an indirect labeling scheme wherein a label is incorporated into the probe in the form of a small molecule such as digoxigenin, fluorescein, or biotin, the probe being capable of specifically binding to the analyte. The label may or may not be detectable on its own and its presence is typically revealed using enzyme conjugates that specifically bind to the small molecule in the probe. For example, in a typical format, the enzyme conjugate is allowed to bind to the small molecule in the probe, and after washing to remove unbound material, a substrate for the enzyme is added. Dioxetane molecules containing a stabilizing group are typically used as the enzyme substrate. In the presence of the conjugate enzyme, the stabilizing group is cleaved, leading to decomposition of the dioxetane, and light emission.

A clear advantage of an indirect labeling scheme is the increased sensitivity one achieves through enzymatic amplification of the signal. However, a disadvantage of such methods as they are currently practiced in the fields is that many steps are required in the assay protocol, requiring more time to complete the assay. Moreover, a greater number of reagents are required which means greater cost. In addition, where the method of detection is enzyme-based, stability of the enzyme and its shelf life need to be considered if one is to expect optimum performance of the assay.

In view of the simplicity of chemical reactions relative to enzymatic reactions, it would be desirable to achieve chemiluminescent signal amplification by a chemical, as opposed to enzymatic means. U.S. Pat. No. 5,516,636 to McCapra and a later publication by Schubert (Nucleic Acids Research, 1995, Vol. 23, No. 22, pg. 4657) describe the use of sensitizer-labeled oligonucleotide probes for the detection of nucleic acid target molecules. A solid phase DNA probe assay is disclosed in which a DNA target molecule is bound to a membrane and hybridized to a sensitizer-labeled oligonucleotide complimentary in sequence to the target DNA. The membrane is subsequently treated with an olefin solution, the olefin being capable of undergoing a chemical reaction upon reaction with singlet oxygen to form a metastable reaction product (dioxetane). Upon exposure of the membrane to ambient oxygen and light, the sensitizer molecules become excited and transfer their excess energy to ambient oxygen for formation of singlet oxygen. The singlet oxygen therein produced reacts with the olefin on the membrane to form a stable 1,2-dioxetane in the area of the hybridization zone, which when subsequently exposed to heat, chemical treat or enzymatic treatment, decomposes to omit light. The use of a sensitizer as a label provides the advantage of amplifying the signal based on repeated excitation/oxygen quenching cycles to achieve a high level of sensitivity.

U.S. Pat. No. 5,800,999 and U.S. Pat. No. 6,063,574, each to Bronstein, describe probes labeled with a dioxetane precursor (olefin) that is reactive with a singlet oxygen produced from either a photochemical, chemical or thermal reaction. In nucleic acid probes, the dioxetane precursor is disclosed as being bound covalently to the probe either through a side chain after formation of the probe, or as part of the sequencing synthesis of the probe. The precursor remains present on the probe throughout hybridization with a target sequence. After washing to remove non-bound material, the dioxetane precursor is photooxygenated, either through the use of a sensitizer suspended in solution, provided with molecular oxygen and visible light, or by intercalating a sensitizer dye after hybridization, followed by irradiation in the presence of molecular oxygen. In either format, singlet oxygen is produced by the sensitizer, and the precursor is photooxygenated to generate a dioxetane. The dioxetane is then caused, or allowed to decompose, emitting light.

U.S. Pat. No. 5,340,714 to Katsilometes describes the binding of a sensitizer (non-metallic tetrapyrrole molecule) to a probe or to an analyte analog. In particular, this patent describes chemiluminescent labeling of an analyte analog which can compete with the analogus analyte (a member of a specific binding pair) for binding to a specific binding pair member. The labeled analyte analog can bind to the specific binding pair member in a manner similar to the analyte. However, the analyte analog is not the substance under detection. A pre-determined amount of the analyte analog must be added to the assay.

Increasingly, nucleic acid amplification based hybridization assay, or in-situ applications are receiving commercial attention. It would be advantageous to provide hybridization assays and in-situ applications which may utilize target amplification techniques, such as PCR, to directly label a target analyte with a sensitizer. Target amplification increases sensitivity by exponentially multiplying the number of copies of target sequences in a sample. The combined benefits of amplifying the target via PCR, for example, and amplifying the signal by the repeated excitation/oxygen quenching cycles associated with the sensitizer label would allow one to achieve an even higher level of sensitivity than has been associated with prior chemiluminescent methods.

### SUMMARY OF THE INVENTION

In one aspect of the invention there is provided a method for detecting the presence of a sensitizer-labeled analyte in a sample. The method includes the step of: (a) exciting a sensitizer label on an analyte; (b) permitting energy from the excited sensitizer label to be transferred to and excite an acceptor molecule, whereby the sensitizer label returns to an unexcited state; (c) reacting the excited acceptor molecule with a chemiluminescent precursor to form a chemiluminescent compound which emits light in response to an activation source; (d) exposing the chemiluminescent compound to the activating source to produce a detectable signal; (e) detecting said signal; and (f) correlating the signal with the presence or absence of the analyte.

A further aspect of the invention is directed to a method for detecting an analyte in a sample, the method including the steps of: (a) immobilizing a sensitizer-labeled analyte on a carrier; (b) exposing the immobilized analyte to light of an appropriate wavelength to electronically excite the sensitizer; (c) permitting energy from the excited sensitizer label to be transferred to and excite an acceptor molecule, whereby the sensitizer label returns to an unexcited state; (d) reacting the excited acceptor molecule with a chemiluminescent precursor to form a chemiluminescent compound which emits light in response to an activation source; (e) exposing the chemiluminescent compound to the activating source to produce a detectable signal; (f) detecting the signal; and (g) correlating the signal with the presence or absence of the analyte in the sample. In particular, this method is useful for both solid phase nucleic acid assays and solid phase immunoassays.

Also provided by the invention is a method for detecting a specific nucleotide sequence in a polynucleotide analyte, the method including the steps of: (a) providing a sensitizer-labeled analyte; (b) providing the specific sequence on a carrier; (c) hybridizing the labeled analyte to the specific sequence, thereby forming a hybridization complex; (d) exposing the hybridization complex to light of an appropriate wavelength to electronically excite the sensitizer; (e) permitting energy from the excited sensitizer label to be transferred to and excite an acceptor molecule, whereby the sensitizer label returns to an unexcited state; (f) reacting the excited acceptor molecule with a chemiluminescent precursor to form a chemiluminescent compound which emits light in response to an activation source; (g) exposing the chemiluminescent compound to the activating source to produce a detectable signal; (h) detecting the signal; and (i) correlating the signal with the presence or absence of the analyte in the sample.

Another aspect of the invention is directed to a method of determining if a patient is at risk for a disorder or has a disorder that includes detecting in a patient specimen the presence or absence of a lesion of an analyte, wherein the detecting includes the steps of: (a) providing a sensitizer-labeled analyte; (b) exciting the sensitizer; (c) permitting energy from the excited sensitizer label to be transferred to and excite an acceptor molecule, whereby the sensitizer label returns to an unexcited state; (d) reacting the excited acceptor molecule with a chemiluminescent precursor to form a chemiluminescent compound which emits light in response to an activation source; (e) exposing the chemiluminescent compound to the activating source to produce a detectable signal; (f) detecting said signal and/or the amount of the signal; and (g) correlating the signal and/or amount of the signal with the presence or absence of the lesion of the analyte in the patient specimen as compared to a control patient specimen.

Further provided by the invention is a system for detecting an analyte including the following components: (a) an analyte labeled with a sensitizer moiety; (b) a chemiluminescent precursor compound capable of forming a chemiluminescent compound which emits light in response to an activation source; and (c) activating source capable of causing the chemiluminescent compound to produce a detectable signal.

Moreover, the invention provides a kit for detecting analyte including: (a) an analyte labeled with a sensitizer moiety; and (b) a chemiluminescent precursor compound capable of forming a chemiluminescent compound which emits light in response to an activation source.

For each of the foregoing inventive aspects, it is the sensitizer bound to the substance to be detected which mediates the chemiluminescent light-producing reaction. The chemiluminescent light is emitted during the time that electronically excited products of chemical reactions return to the ground state.

### BRIEF DESCRIPTION OF THE DRAWINGS

FIG. 1 shows the reactions involved in the sensitizer-catalyzed generation of singlet oxygen.

FIG. 2 shows the reaction of singlet oxygen with a chemiluminescent olefin to form a 1,2-dioxetane.

FIG. 3 shows the induced decomposition of the 1,2-dioxetane formed in FIG. 2 by an appropriate trigger to release light. Preferred triggering conditions include a change in pH or temperature.

FIG. 4 shows chemiluminescent signal amplifications by a sensitizer means, the sensitizer being directly bound to the analyte (substance to be detected). The sensitizer allows for amplification of the signal based on repeated excitation/oxygen quenching cycles to achieve a high level of sensitivity.

FIG. 5 shows solid phase detection of immobilized target nucleic acid which has been labeled with a sensitizer. The target DNA labeled with the sensitizer may be directly bound to the membrane or may be hybridized to a specific sequence that has been bound to the membrane.

FIG. 6 shows the synthesis of sensitizer-labeled dUTP.

FIG. 7 shows the 5-′ labeling of an aminofunctionalized oligonucleotide primer.

### DETAILED DESCRIPTION OF THE INVENTION

As defined herein, the term analyte refers to the compound or composition to be detected. The analyte may be a peptide, PNA (peptide nucleic acid) polypeptide, protein, oligonucleotide, polynucleotide, antibody, antigen, ligand, receptor, hapten, saccharide or polysaccharide. Furthermore, the analyte can be a part of a cell, such as a bacteria or a cell bearing a blood group antigen or an HLA antigen or a microorganism.

Sensitizer label, photosensitizer label, and the like as defined herein is a substance directly bound to the analyte, which when exposed to suitable conditions causes light to be produced. In particular, when appropriately combined with molecular oxygen and light of an appropriate wavelength or any other electric or electromagnetic excitation and a chemiluminescent precursor, the sensitizer label causes light to be produced. In one sense, a sensitizer can be a molecule with a chromophore that is capable of absorbing light so that it becomes electronically excited.

The term chemiluminescence, chemiluminescent and the like refers to the production of light by way of a chemical reaction. It may further be defined as the light emitted during the time that electronically excited products of chemical reactions return to the ground state.

A member of a specific binding pair refers to one of two different molecules having an area on the surface or in a cavity which specifically binds to and is, thereby, defined as complimentary with a particular spatial and polar organization of the other molecule.

Specific binding and the like refers to the specific recognition of one of two different molecules for the other compared to substantially less recognition of other molecules. Exemplary of specific binding are antibody-antigen interactions, enzyme-substrate interactions, polynucleotide interactions, and so forth.

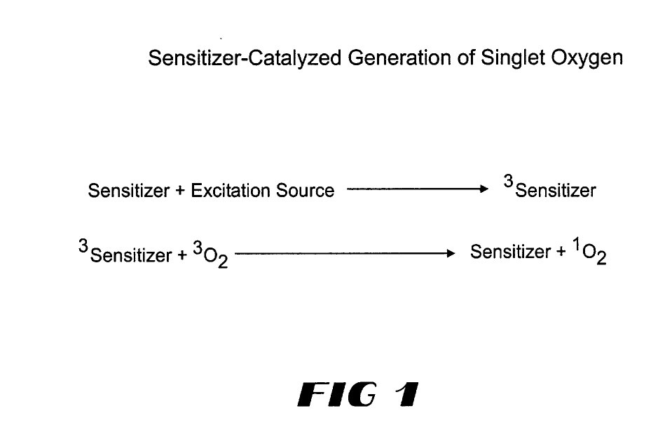

The inventive aspects of the present invention are achieved by the provision of analytes bearing a sensitizer label bound directly thereto. In preferred embodiments of the inventive methods, a sensitizer provided with molecular oxygen and light of an appropriate wavelength may produce singlet oxygen in accordance with the reactions shown in FIG. 1. The sensitizer will assume an excited triplet state upon excitation by exposure to a suitable wavelength of light. The sensitizer interacts with an acceptor molecule. In one desired embodiment, the acceptor molecule is molecular oxygen in the ground state. The photosensitizer in its triplet state (excited state) is capable of converting ground-state oxygen (a triplet) to an excited singlet state, the singlet oxygen capable of resulting in the production of the detectable signal which can be monitored. In one embodiment of the methods of the present invention, the amount of signal produced is measured, wherein the amount of the signal is correlated to the amount of analyte present in a given sample.

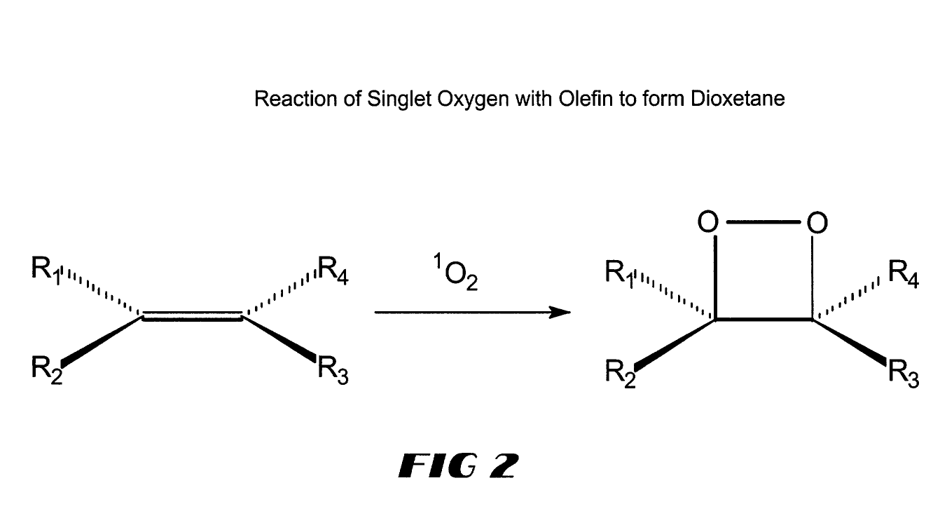

In preferred embodiments of the present invention, the singlet oxygen produced as shown in FIG. 1 reacts with an olefin to form a dioxetane. In particular, the singlet oxygen may react by a 1,2-cycloaddition with an olefin to give a 1,2-dioxetane, as shown in FIG. 2. The dioxetane formed is a metastable reaction product, which is capable of decomposition with the simultaneous or subsequent emission of light, usually within the wavelength range of 250 to 1,200 nm.

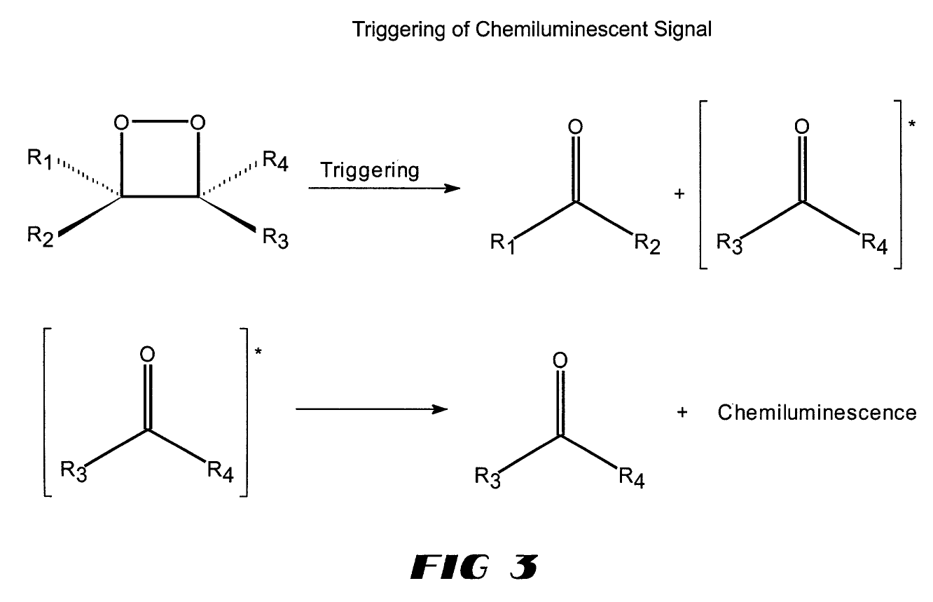

It is noted that, for the olefin and metastable dioxetane shown in FIGS. 2 and 3, definitions of suitable R substituents can be found in, but are not limited to, those in U.S. Pat. No. 5,386,017. For example, R₁ may be selected from alkyl, alkoxy, aryloxy, dialkyl or aryl amino, trialkyl or aryl silyloxy groups and R₂ is an aryl group substituted with an X oxy-group, wherein the 1,2 dioxetane forms an unstable oxide intermediate 1,2-dioxetane compound when triggered to remove X by an activating agent so that the unstable 1-2, dioxetane compound decomposes to form light and two carbonyl-containing compounds (shown in FIG. 3) wherein X is a labile group which is removed by the activating agent to form the unstable oxide intermediate and wherein R₃ and R₄ are selected from aryl and alkyl groups which can be joined together as spirofused polycyclic alkyl and polycyclic aryl groups.

Referring now to FIG. 3, some dioxetanes decompose by heating, chemical, electrical, electrochemical, electrostatic, or enzymatic means to produce light. For example, the 1,2-dioxetane shown in FIG. 3 may be cleaved thermally to carbonyl-containing products.

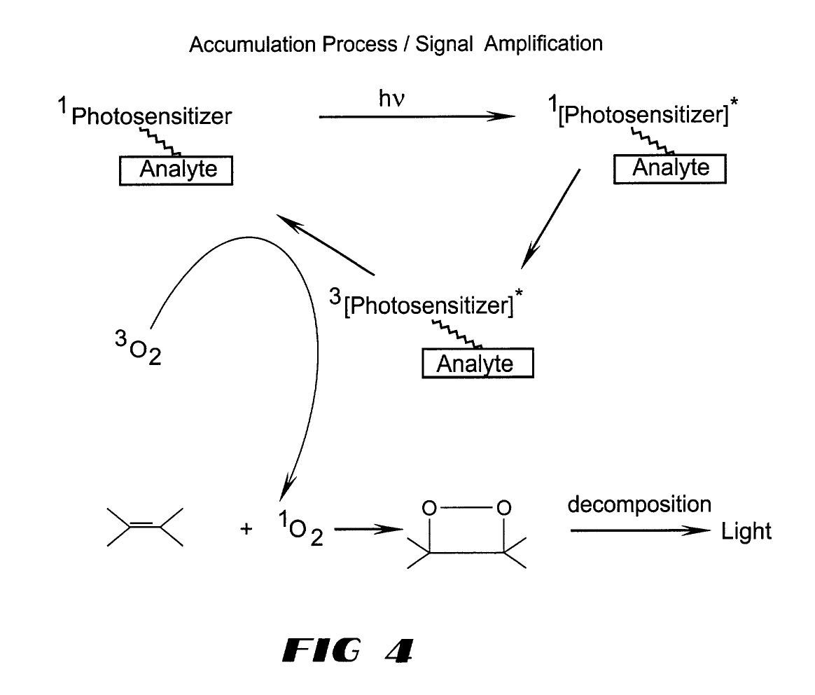

In commercial assay systems, the label typically forms a signal directly by such means as a color change, emission of light or radiation. Generally speaking, the intensity of the signal of a chemiluminescent compound in such systems is correlated with the amount of chemiluminescent compound present, as well as the chemiluminescent efficiency of the chemiluminescent compound. Only a finite signal intensity can be generated that is correlated with the amount of label attached to a probe, for example, and the efficiency of the association of the probe to bind directly or indirectly to the analyte. In contrast, the present invention has the advantage of allowing for a much larger amount of unlabeled chemiluminescent compound (for example, dioxetane) to be produced in the assay. This is illustrated in FIG. 4. Since the acceptor molecule of the energy, shown as molecular oxygen in FIG. 4, is present in great excess over the sensitizer label, the continuous recycling of the sensitizer during irradiation by the exciting light, will lead to amplifications several fold over the concentration of the label. The signal is created as a result of the donor-acceptor interaction between the excited triplet state sensitizer and the acceptor molecule (ground-state molecular oxygen). The sensitizer is allowed to return to its original state after it has passed its energy to the acceptor. Preferably, this occurs by a triplet-triplet annihilation. Because the sensitizer is still present in association with ground-state oxygen, it is available for another excitation, followed by energy transfer to the acceptor for the production of an even greater signal. This type of excitation and energy transfer may be repeated many times within a very short period of time so that the use of a sensitizer as a label on the analyte provides the added advantage of amplifying the signal, and thus increasing the sensitivity of the assay.

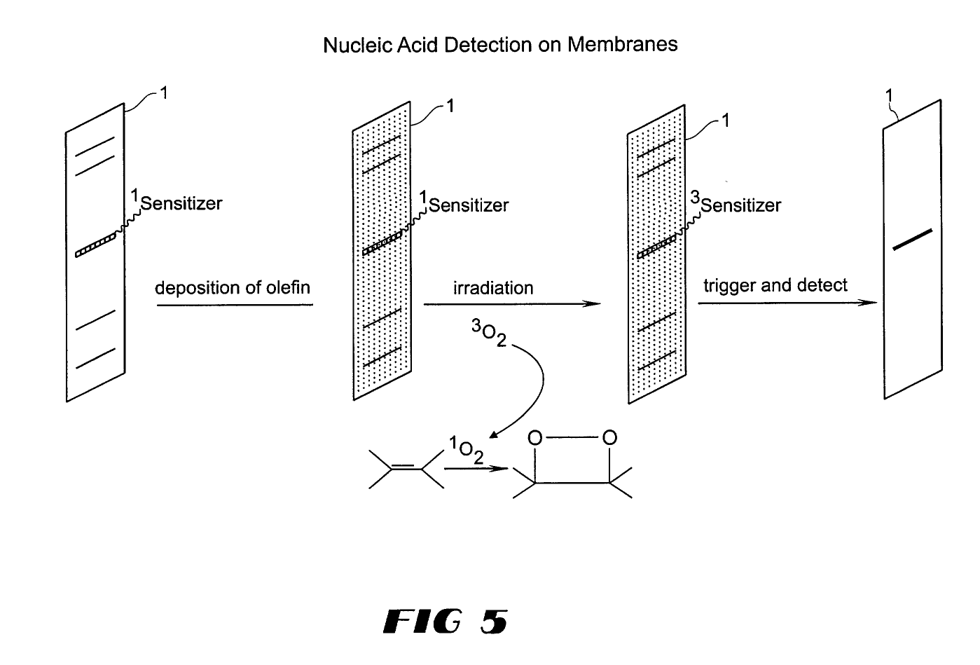

With reference now to FIG. 5, in one embodiment sensitizer-labeled analyte may be immobilized directly on membrane 1 for detection. Alternatively, labeled analyte may be immobilized to a given membrane by binding to a specific sequence on the membrane. As shown in FIG. 5, once the target analyte, such as DNA, has been immobilized to membrane 1, olefin may be deposited on the membrane by such means including, but not limited to, dipping, soaking, painting, spraying, pipetting or spotting the olefin on the membrane. Following irradiation of an appropriate wavelength, such as 670 nm for methylene blue, the sensitizer becomes electronically excited and transfers its excess energy to ground-state oxygen for the production of a singlet oxygen. The singlet oxygen therein produced reacts with the olefin on the membrane to form a stable 1,2-dioxetane in the area corresponding to the analyte zone, which when subsequently exposed to heat, chemical treatment or enzymatic treatment decomposes to emit light. In one embodiment, the 1,2-dioxetane is exposed to chemical treatment with a base at a pH of about 11.0. In a preferred embodiment, the 1,2 dioxetane is triggered by heating to a temperature of about 60° to about 100° C. As shown, the signal may be detected in the form of a band on X-ray film. In an additional embodiment, the light energy produced may be detected by means of a photoelectric cell. Although the signal may be detected optically, it is preferred that the signal is recorded by means of a light-sensitive film, photoelectric cell, or other suitable means.

In a preferred embodiment, the labeled analyte is immobilized on a carrier such as a membrane or a gel. The carrier may be a particle, such as a bead, film, membrane, microtitre or other type well, strip, and the like. Examples of some suitable carrier compositions include nylon, nitrocellulose, polyacrylamide, polyacrylate, poly(vinylfluoride), polystyrene, polypropylene, glass, or metal, alone or in combination with other materials.

Binding of the labeled analyte to the carrier may be direct or indirect, covalent or non-covalent. Desirably, the labeled analyte is bound either directly or indirectly via covalent interactions to the carrier. For example, as shown in FIG. 5, labeled nucleic acid may be spotted directly on a membrane for detection. Alternatively, a specific nucleic acid sequence may be bound to the membrane and used to capture a labeled target analyte complimentary in sequence to the specific sequence on the membrane. In this way, the target analyte may be bound in an indirect fashion to a solid phase for detection.

In this regard, it is well within the contemplation of the present invention that target DNA that has been random-primed labeled with a sensitizer may be hybridized to specific probe sequences that have been immobilized to a membrane. The presence of a hybridization signal may correlate with the presence of the specific sequence within the target DNA. This method may be useful for deciphering the presence (or absence) of a mutation within a given target nucleic acid sample.

As described above, the analyte is the substance to be detected. In one embodiment, this substance may be selected from the following: polynucleotide, protein, peptide, polypeptide, PNA (peptide nucleic acid), hapten, saccharide, polysaccharide, antigen and antibody. Polynucleotide analytes include, but are not limited to, DNA (single-stranded or double-stranded), DNA-RNA duplexes, m-RNA, r-RNA, and t-RNA. The analyte under detection may also include substances which are capable of binding to polynucleotides, such as including, but not limited to, enzymes, activators, repressors, repair enzymes, polymerases, and nucleases. The analyte may be found directly in a sample from a patient, such as a biological tissue or body fluid. The sample can either be directly used or may be pretreated to render the analyte more detectable.

There is a need in the art to develop labels for nucleic acid detection that can provide resistance to harsh hybridization conditions and high sensitivity, and that can result in reliable emission of light in nucleic acid assays. Increasingly, PCR technology has been used commercially in probe hybridization assays, as well as in-situ applications. Enzyme labels, which are required for use of many of the enzyme-cleavable dioxetanes presently in use commercially, may not always be appropriate for such assays. In particular, most enzymes cannot withstand the harsh conditions typically used in processing nucleic acids, such as high temperatures and organic or inorganic solvents. The present invention satisfies a need in the art by providing assays that employ a sensitizer label that may be incorporated by such means as PCR amplification within the analyte, the label being able to provide high sensitivity while withstanding harsh nucleic acid processing conditions.

In one embodiment of the methods of the present invention, a polynucleotide analyte may be labeled by incorporation of a sensitizer-labeled nucleotide during a nucleic acid amplification reaction, primer extension reaction, or an in vitro transcription reaction. In a further embodiment, a polynucleotide analyte may be labeled by incorporation of a sensitizer-labeled primer during a target amplification reaction, primer extension reaction or an in vitro transcription reaction. The primers may be either random or specific primers. These reactions are described by Ausubel, F. M. et al. (Eds.), Current Protocols in Molecular Biology, John Wiley & Sons, Inc., New York (1999).

Table 1 below shows labeling strategies for incorporating a sensitizer-labeled nucleotide within the target nucleic acid. Furthermore, Table 1 shows that an aminofunctionalized nucleic acid may be labeled by reaction with an NHS ester form of a sensitizer.

TABLE I
Labeling Strategies For Nucleic Acids

| Procedure | Labeled Compound |
|---|---|
| Incorporation of Labels by PCR | dUTP, primers |
| Random Primed DNA Labeling | dUTP, hexamers |
| Labeling of RNA with RNA polymerase | UTP, primers |
| (NASBA) | |
| Labeling by Nick Translation | dUTP |
| 3′-Labeling of ssDNA with Terminal Transferase | (d) dUTP |
| Labeling of Aminofunctionalized Nucleic Acids | NHS Ester |

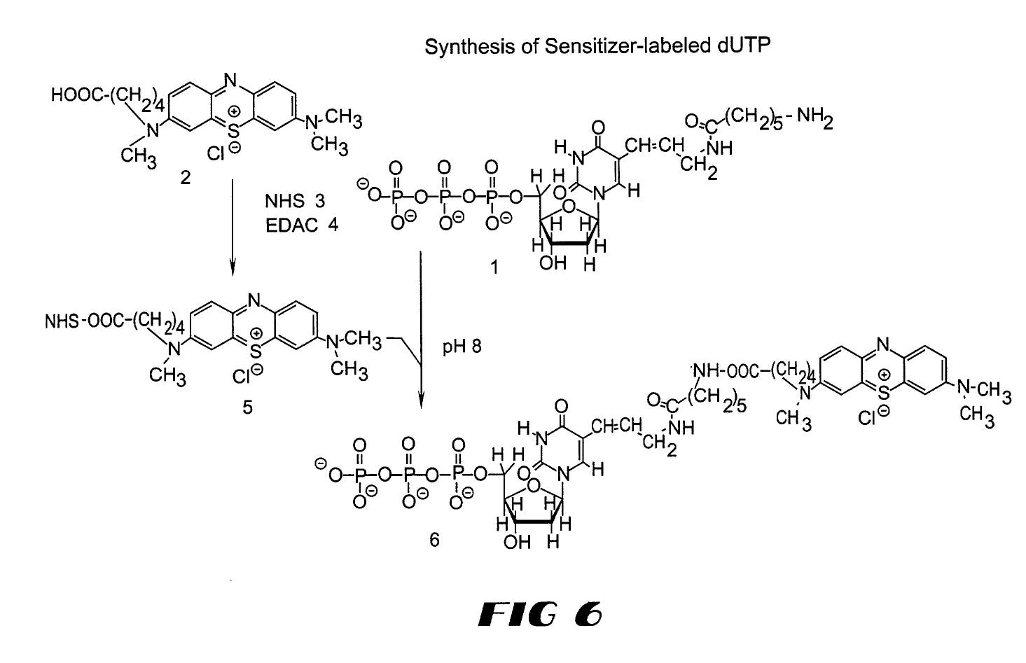

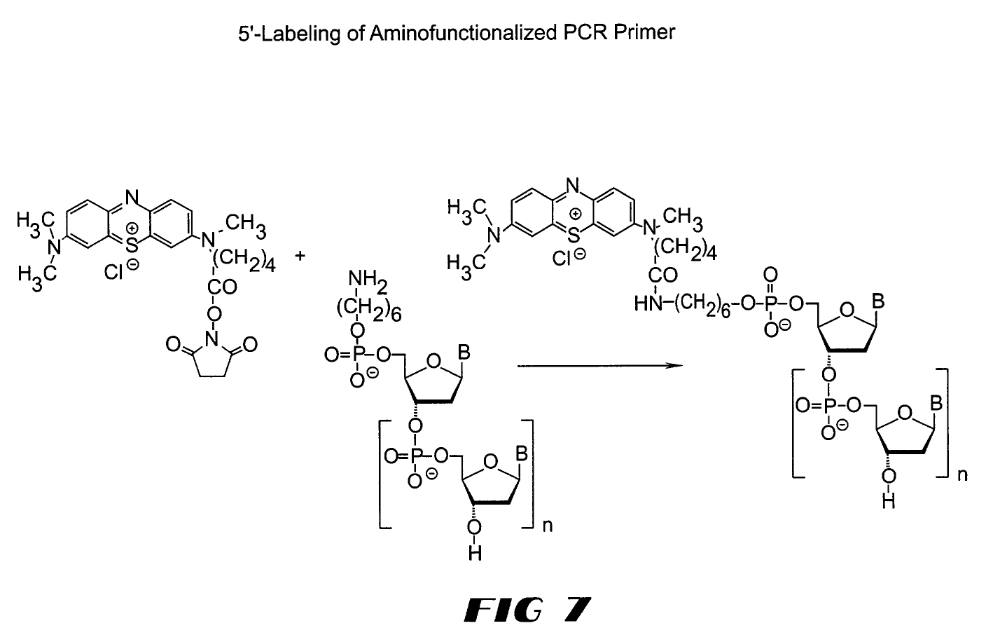

We refer now to FIG. 6, which shows the synthesis of a sensitizer-labeled dUTP. In particular, the methylene blue sensitizer (compound 2) is reacted with N-hydroxysuccinimide (compound 3) and EDAC: 1-ethyl-3-(3-dimethylamino propyl) carbodiimide HCl (compound 4) to form the activated ester form of the sensitizer (compound 5). The activated ester form is reacted with aminofunctionalized dUTP (compound 1) at pH 8 to form the sensitizer-labeled dUTP (compound 6), which may be incorporated as a building block within an analyte to be detected. Moreover, as shown in FIG. 7, an N-hydroxysuccinimide ester form of methylene blue may be reacted with an aminofunctionalized oligonucleotide primer in order to obtain a 5′-labeled primer useful for PCR incorporation of the label within the analyte.

It is well within the contemplation of the present invention that various methods may be used to incorporate the sensitizer label within the analyte. By way of example, in desired embodiments of the invention, the polynucleotide analyte may be labeled by incorporation of a sensitizer-labeled nucleotide or primer during a target amplification reaction selected from the group consisting of PCR, RT-PCR, NASBA, LCR, SAGE, and differential display, as well as combinations thereof. Such techniques are well known in the art.

For example, polymerase chain reaction (PCR) is a method for in vitro amplification of a segment of DNA described by Saiki, et al. in Science 239: 487 (1988), Mullis et al. in U.S. Pat. No. 4,683,195, Ausubel, F. M. et al. (Eds.), Current Protocols in Molecular Biology, John Wiley & Sons, Inc., New York (1999), and Wu, R. (Ed.), Recombinant DNA Methodology II, Methods Enzyrnol., Academic Press, Inc., New York, (1995). In general, a PCR reaction contains template DNA with the target sequence to be amplified, two primers complementary in sequence to the target DNA, nucleotides, buffer, and a thermostable DNA polymerase. The reaction mixture is subjected to several cycles of incubation at temperature for denaturation, annealing and elongation, resulting in exponential amplification of the target DNA. The oligonucleotides primers may be synthesized by methods known in the art. Suitable methods include those described by Caruthers in Science 230:281-285 (1985) and DNA Structure, Part A: Synthesis and Physical Analysis of DNA, Lilley, D. M. J. and Dahlberg, J. E. (eds), Methods Enzymol., 211, Academic Press, Inc., New York (1992). The amplified fragment may be cloned, sequenced and may be further amplified to obtain a longer nucleic acid molecule.

Furthermore, RT-PCR (reverse transcriptase-polymerase chain reaction) is a method of amplifying first-strand cDNA products by the polymerase chain reaction. (Ausubel, F. M. et al. (Eds.), Current Protocols in Molecular Biology, John Wiley & Sons, Inc., New York, (1999)). This method involves a two-step process in which mRNA is used as a template to synthesize first-strand cDNA using a reverse transcriptase with either a gene-specific or traditional cDNA synthesis primer. The resulting first-strand cDNA is then amplified by PCR, generating many copies of a targeted DNA.

NASBA (see EP 0329822) is a method used for the amplification of RNA. In particular, it is a specific, isothermal method of nucleic acid amplification that involves the coordinated activities of three enzymes, AMV reverse transcriptase, RNaseH, and T7RNA polymerase. Quantitative detection is achieved by way of internal calibrators, which are added at isolation, which are coamplified and subsequently identified along with the wild-type of RNA using a suitable means such as electrochemiluminescence.

The ligase chain reaction (LCR) is a DNA amplification technique which can be used to detect trace levels of known nucleic acid sequences (Landegren, U., et al. (1988) Science 241; 1077-1080 and Barany, F. (1991) PCR Methods and Applications Journal; 1:5-16). This method involves a cyclic two-step reaction: (1) a high-temperature melting step in which double-stranded target DNA unwinds to become single-stranded, and (2) a cooling step in which two sets of adjacent, complimentary oligonucleotides anneal to the single-stranded target molecules and ligate together. The products of the ligation from one cycle serve as templates for the next cycle's ligation reaction. The LCR technique results in the exponential amplification of the ligation products in a manner analogous to PCR amplification.

Differential display has been described by P. Liang and A. Pardee in Science 257: 967-971 (1992) and P. Liang et al. in Nucleic Acids Research, 22, No. 25: 5763-5764 (1994). The general strategy of this method is to amplify partial cDNA sequences from subsets of mRNAs by reverse transcription and the polymerase chain reaction (PCR). A key element in this method is to use a set of oligonucleotide primers, one being anchored to the polyadenylate tail of a subset of mRNAs, the other being short and arbitrary in sequence such that it anneals at different positions relative to a first primer. The mRNA subpopulations that are defined by these particular primer pairs are amplified after reverse transcription and then resolved on a DNA sequencing gel. Differential display is useful to identify and isolate those genes that are differentially expressed in various types of cells or under altered conditions.

Serial analysis of gene expression (SAGE) has been described by V. Velculescu et al. in Science 270: 484-487 (1995). This technique allows for the quantitative and simultaneous analysis of a large number of transcripts. In this technique, in order to amplify cDNAs of unknown sequence, one uses a modified random primer. In particular, the random primer (an oligonucleotide with random sequence, usually either a hexamer or nonamer) is modified by combining it with a poly A or other anchoring sequence. A brief overview of the SAGE technique is as follows. Double-stranded cDNA is synthesized from mRNA by means of a biotinylated oligo (dT) primer. The cDNA is then cleaved with a restriction endonuclease (anchoring enzyme) that would be expected to cleave most transcripts at least once. The 3′ portion of the cleaved cDNA is then isolated by binding to streptavidin beads. This allows for the production of a unique site on each transcript that corresponds to the restriction site that is located closest to the polyadenylate tail. An adapter containing a type IIS restriction site is then ligated to these fragments, where the type IIS restriction endonuclease cleaves at a defined distance up to 20 base pairs away from their asymmetric recognition site. Cleavage with the type IIS restriction endonuclease results in short expression sequence tags (EST) from a defined position within each of the mRNA transcripts present in the original tissue. These fragments are then ligated together to form a concatemer of EST's which are cloned into a standard plasmid vector and sequenced. Each clone can contain identifying tags for up to 60 mRNA transcripts.

According to the present invention, a useful sensitizer or photosensitizer is any label directly bound to the analyte that, when excited by radiation of a particular wavelength or other chemical or physical stimulus, can achieve an excited triplet state. In one preferred embodiment, the sensitizer is a dye. The dye may be selected from the following: methylene blue, porphyrins, metalloporphyrins, aromatic hydrocarbons, pyrenes, phthalocyanine, hemin, flavin derivatives, rhodamine heterocyclic compounds, xanthine, tri-aryl methane dyes, phenothiazinium dyes, and acridinium dyes.

As described above, sensitizers may be linked to the analyte by methods which are well known in the art, including by use of one or more functional groups chemically bound to the sensitizer that react with a complimentary functional group associated with the analyte or a building block of the analyte, such as a nucleotide or PCR primer. For example, with further reference to FIG. 6, a sensitizer dye may be bound to an analyte using a functional group such as a N-hydroxysuccinimidyl ester linker to react with a complimentary amine linking group to allow for incorporation of the sensitizer via an amide group into a nucleotide of an analyte. Alternatively, an N-hydroxysuccinimidyl ester linker may react with a thiol or hydroxy linking group to incorporate the sensitizer via a thiol ester or ester group into the building block of an analyte. In one preferred embodiment, the sensitizer is first incorporated into the building block and the building block is thereafter incorporated within the analyte. For example, one may incorporate a sensitizer-labeled dUTP or other deoxynucleotide into analyte DNA via PCR incorporation into the analyte. Alternatively, as shown in FIG. 7, one may use a sensitizer-labeled PCR primer to incorporate the sensitizer label within the analyte. In this example, a N-hydroxysuccinimidyl ester linker reacts with the aminofunctionalized PCR primer.

In preferred embodiments of the present invention, the chemiluminescent precursor is an olefin. Preferred olefins will have electron donating groups among their substitutions so as to produce dioxetanes with increased quantum yield upon decay. Furthermore, it is well within the contemplation of the present invention that the olefin also contain a fluorescent moiety for an additional increase in quantum yield from the resulting dioxetane decomposition. Exemplary of electron rich olefins suitable as chemiluminescent precursors in the methods of the present invention are the following: enol ethers, enamines, 9-alkylidene-N-alkylacridans, arylvinylethers, 1,4-dioxenes, 1,4-thioxenes, 1,4-oxazines, arylimidazoles, 9-alkylidene-xanthenes and lucigenin. The electron donating group or groups in the olefin should be at a position that increases the reactivity of the olefin to singlet oxygen and/or imparts fluorescence to the product of disassociation of the resultant dioxetane. The electron donating group may be, for example, hydroxyl, alkoxy, di-substituted amino, alkyl thio, furyl, pyryl, halogen, and so forth. In one example, an enol ether may have at least one aryl group bound to the olefinic carbons where the aryl ring is substituted with an electron donating group at a suitable position so as to increase the reactivity of the olefin to singlet oxygen and/or impart fluorescence to the product of disassociation of the resulting dioxetane.

Desirably, these chemiluminescent olefins will emit light at a wavelength above 300 nanometers, preferably above 500 nanometers, and more preferably, above 550 nanometers. Most preferably, the chemiluminescent olefin will emit light at a wavelength beyond the region where components of the sample contribute in a significant way to light absorption.

Olefins having the structure shown below have been described in U.S. Pat. No. 5,386,017 to Schaap.

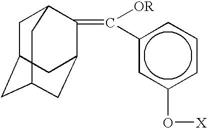

These olefins are suitable for practice of the present invention. However, the invention is not limited to these olefins. Treatment of a stable dioxetane with an appropriate activating agent produces chemiluminescence. The X group on the dioxetane represents a labile leaving group. This group may be activated or chemically cleaved by chemical means in one example. Examples of typical X groups which can be removed chemically, as well as enzymatically are shown in U.S. Pat. No. 5,795,987. Useful X-oxy protecting groups include, but are not limited to, hydroxyl, alkyl or aryl, carboxyl ester, inorganic oxy-acid salt, alkyl or aryl silyloxy and oxygen pyranocide. Additional examples of protecting groups, as well as the corresponding cleavage/activating agents useful for removal of X can also be found in the standard treatise on protecting groups (Greene and Vuts, in Protective Groups in Organic Synthesis, 1999).

In one embodiment, the dioxetane decomposes spontaneously. In another embodiment, the dioxetane is caused to decompose by an appropriate activating source, such as chemical means. For example, the activating source may be a base and/or heat. The base may be a solid chemical component which is incorporated into a carrier. For example, co-pending, commonly owned U.S. patent application Ser. No. 09/913,653 describes the use of an activating film for use in chemiluminescent assays that include at least one solid chemical component immobilized on or impregnated therewith which when acted upon by an energy source, such as heat, releases an activating substance. This activating substance in the presence of the chemiluminescent precursor (for example, the chemiluminescent olefin) reacts therewith to produce a chemiluminescent signal for the detection of a target molecule. The disclosure of this application is incorporated herein by reference.

In a further embodiment of the present invention, the chemiluminescent precursor may be present as a solid chemical component on a carrier. For example, co-pending, commonly owned U.S. patent application Ser. No.______ discloses a film component for use in chemiluminescent assays that includes a solid film substrate and at least one chemiluminescent precursor component immobilized therewith which produces a triggerable chemiluminescent compound, the film being free of compounds which generate singlet oxygen and being adapted for use with a sensitizer-labeled analyte or agent probative of the analyte. The disclosure of this co-pending application is incorporated herein by reference.

The invention is further demonstrated by the following illustrative examples, which are not intended to limit the invention.

### EXAMPLE 1

Preparation of Aminoreactive Photosensitizer

An activated N-hydroxysuccinimide ester form of a methylene blue sensitizer was obtained according to procedures described by Motsenbocker et al. Photochem. Photobiol., 58, 648-652 (1993).

### EXAMPLE 2

Preparation of Functionalized dUTP and Oligonucleotides

Aminofunctionalized dUTP was purchased from: Molecular Probes, Eugene, Oreg., USA.

The 5′-aminomodified oligonucleotides, carboxyfunctionalized as well as unmodified oligonucleotides described in the examples below were synthesized on a PE Biosystems Nucleic Acid Synthesizer, Model No. ABI 3948 using methods well known in the art.

### EXAMPLE 3

Detection of Sensitizer-Labeled Target Nucleic Acid

Sensitizer-labeled nucleic acid was spotted on a Hybond+nylon membrane (Amersham Biosciences Corporation), along with negative controls at various concentrations ranging from 25 to 500 fmoles in a total volume of 1 μl. The positive control consisted of 1 μl of 100 fmoles of dicarboxyl methylene blue dye (EMP Biotech, Berlin, Germany). After spotting, the membrane was dried at 65° C. for 10 minutes, followed by dipping the membrane in an olefin solution (100% w/v in hexane or methanol) wherein olefin was synthesized by the method of Schaap as described in U.S. Pat. No. 4,857,652 and allowing it to air dry, then illuminating the spotted surface with red light for 15 minutes to excite the sensitizer dye and form a triggerable dioxetane. In order to detect the signal, the dioxetane was first triggered. In one format, a sheet of filter paper previously soaked in a saturated solution of ammonium carbonate and then dried to a solid form was taped to a glass plate. The hybridized membrane with bound target DNA was subsequently placed (DNA side up) on top of the filter paper containing the dried base and a sheet of plastic was placed on top of this. In the dark, a sheet of Hyperfilm ECL (Amersham Biosciences Corporation) was placed over the plastic sheet and another glass plate was placed on top. The whole sandwich formation was incubated at 80° C. for 15 minutes to allow for release of the base from the filter paper and resultant activation of the dioxetane present on the hybridized membrane. Alternatively, a sandwich formation was prepared as described, except without the filter paper containing the base, and the dioxetane was triggered by heating to above 100° C. For either format, the film was developed using standard techniques and successful hybridization was observed as black spots on the Hyperfilm ECL with the lowest quantity of DNA detected being in the range of 25 fmoles.

### EXAMPLE 4

Labeling of 5-aminoalkyl-dUTP

A solution was formed by dissolving 5 mg (7 μmol) of 5-aminoalkyl-dUTP in 4 mL of 0.1 M phosphate buffer pH 8 and 500 μL dimethylformamide. To this solution was added a solution of 21 μmol aminoreactive sensitizer in dimethylformamide and this reaction mixture was slowly shaken for two hours in darkness at room temperature. The mixture was centrifuged and the precipitate discarded.

The supernatant from this reaction was separated by gel filtration on a Biogel P2 column (40×16 cm) and the product fraction (first peak) with water as elutant was isolated. The aqueous phase was evaporated on a rotary evaporator at 40° C. and the final product purified in a second step by HPLC on a RP18-column (Nucleosil 120, 120×16 mm) using a linear gradient of 10% buffer B (0.1 M triethylammonium acetate, pH 7.5, acetonitrile, 5/95, v/v) to 40% in buffer A (0.1 M triethylammonium acetate, pH 7.5) over 30 minutes. The sodium salt of the labeled dUTP was obtained by dissolving the product in methanol (0.5 mmol in 5 mL) and adding 15 equivalent of sodium perchlorate solved in dry acetone (five times volume of methanol). The precipitate formed was centrifuged and washed with acetone. The product was dried and stored at −20° C.

### EXAMPLE 5

Labeling of DNA via PCR Incorporation of Labeled-dUTP

A sufficient amount of a PCR master mix was prepared so as to yield the following volumes/amounts per PCR tube: 28 μl H₂O, 5 μl 10×PCR buffer (10×PCR buffer=10 mM Tris HCl, pH 8.3, 500 mM KCl), 4 μl of 25 mM MgCl₂ (2 mM final concentration), 1 μl of 20 μM sense primer (0.4 μM final concentration), 1 μl of 20 μM antisense primer (0.4 μM final concentration), 5 μl of mix of 2 mM each of: dCTP, dATP, dGTP (0.20 mM final concentration of each), 5 μl of mix of TTP (1.6 mM) and labeled dUTP (0.4 mM) for final concentration of 160 μM TTP and 40 μM labeled dUTP, and 0.5 μl of 5 units/μl Taq DNA polymerase (final 2.5 units per PCR tube).

After addition of 0.5 μL of purified DNA template (typically 0.1 to 1 μg dissolved in water or a suitable buffer) per PCR tube, 49.5 μL of PCR master mix above were aliquoted to each tube to bring the final volume of each reaction to 50 μL. Each tube was vortexed and centrifuged briefly. The tubes were placed into a thermal cycler and the cycling started according to the following schedule:

| | | | |
|---|---|---|---|
| first denature | 2 | min | 94° C. |
| denature per cycle | 30 | sec | 94° C. |
| annealing | 30 | sec | on dependency of the primers |
| extension | 1 | min | 72° C. |
| 29 cycles | | | |
| time delay | 10 | min | 72° C. |
| soak | 10 | min - ∞ | 4° C. |

To remove excess sensitizer-labeled dUTP following amplification, the reaction mixture was passed through a Centri-Sep column (Princeton Separations, Inc., Adelphia, N.J.) previously hydrated with Tris-Cl (pH 8.5) buffer. The amplified PCR samples were stored at 4° C. until use. Labeled analyte was detected by the method of Example 3.

### EXAMPLE 6

Labeling of DNA Following PCR Incorporation of Unlabeled Aminofunctionalized Nucleotide

Using a PCR protocol similar to that described in Example 5, unlabeled aminoalkylfunctionalized dUTP was incorporated into DNA by PCR. Following incorporation, the aminolabelled DNA is purified by gel filtration (Centri Sep 40, Princeton Separations, Inc., Adelphia, N.J.), or ethanol precipitation according to known methods to remove free residual aminoalkyl-dUTPs, which will disturb the following labeling reaction.

Two μg amino-modified DNA are dissolved in 3 1L water and 5 μL of 0.25 M sodiumbicarbonate buffer (pH 8) are then added. Afterwards, 5 μL of a solution containing 5 μmol succinimidyl ester of the photosensitizer in 100 μL of DMSO is added to the DNA solution. The reaction mixture is vortexed and allowed to stand for 2 hours at room temperature. Separation of the labeled PCR fragment from the excess dye is achieved by gel filtration or ion-exchange chromatography. For example, the separation may be achieved by passing an aliquot of the reaction mixture through a Centri-Sep column (Princeton Separations, Inc., Aldelphia, N.J.) previously hydrated with Tris-Cl (pH 8.5) buffer according to directions of the manufacturer. The labeled PCR products were detected by the method of Example 3.

### EXAMPLE 7

Labeling of 5′-Aminofunctionalized Primers

The sodium salt of a 5′-aminofunctionalized oligonucleotide (10 nmol) was dissolved in 40 μL of 0.1 M sodiumbicarbonate buffer (pH 8). To the oligonucleotide solution was added 10 μL of a solution containing 0.5 μmol of the succinimidyl ester of the photosensitizer in 100 μL of DMSO. The reaction mixture was vortexed and allowed to stand for 2 hours at room temperature. Separation of the labeled oligonucleotide from the excess dye was achieved by gel filtration, butanol extraction or purified by HPLC according to methods well known in the art.

### EXAMPLE 8

Labeling of PCR Products with Labeled Primers

Labeled oligonucleotides prepared as described in Example 7 were incorporated into target DNA by the following method.

A sufficient amount of a PCR master was prepared so as to yield the following volumes/amounts per PCR tube: 34 μl H₂O, 5 μl 10×PCR buffer, 4 μl of 25 mM MgCl₂ (2 mM final concentration), 0.5 μl of 50 μM 5′-labeled sense primer (0.5 μM final concentration), 0.5 μl of 50 μM antisense primer (0.5 μM final concentration), 5 μl of mix of 2 mM each of: dCTP, dATP, dGTP, dTTP (0.20 mM final concentratiion of each), and 0.5 μl of 5 units/μl Taq DNA polymerase (for final 2.5 units per PCR tube).

After addition of 0.5 μL of purified DNA template (0.1-1.0 μg) dissolved in water or a suitable buffer per PCR tube, 49.5 μL of PCR master mix were aliquoted to each tube to bring the final volume of each reaction to 50 μL. Each tube was vortexed and centrifuged briefly. The tubes were placed into the thermal cycler and the cycling started according to the same schedules described in Example 5.

The PCR samples were stored at 4° C. until use. Thereafter, the labeled PCR products were detected according to the method of Example 3.

### EXAMPLE 9

Labeling of Target RNA Using NASBA Technology

Per reaction, 6 μL of sterile water, 4 μL of 5×primer mix (5×primer mix=1 mM each of antisense and sense primers in 75% DMSO), 4 μL of 5×reaction buffer (5×reaction buffer=200 mM Tris-HCl, pH 8.5, 60 mM MgCl₂, 350 mM KCl, 2.5 mM DTT, 5 mM of dATP, dGTP and dCTP, 4 mM TTP, 1 mM labelled dUTP, 10 mM each of ATP, UTP and CTP, 7.5 mM GTP and 2.5 mM ITP) are added into a microtube. After addition of 1 μL of input material (RNA or purified virus) per reaction (0.02 to 1 μg), the tubes are vortexed and centrifuged briefly. Then, the reaction mixtures are incubated at 65° C. for 5 min and, after cooling to 41° C. for 5 min, 5 μL of enzyme mix (enzyme mix=375 mM sorbitol, 420 μg/mL BSA, 16 U/mL RNase H, 6400 U/mL T7 RNA polymerase and 1280 U/mL AMV-reverse transcriptase) are added. Reactions are then incubated at 41° C. for 90 min and amplificates are stored at −20° C. for further use. The labeled target RNA was detected by the method of Example 3.

### EXAMPLE 10

Labeling Target DNA by Use of Terminal Transferase

Ten pmoles of target DNA (with free 3′-OH groups in 5 μl of EDTA—free buffer or distilled water was mixed on ice with the following terminal transferase reaction mixture: 21.5 μl H₂O, 10 μl of 5×PCR reaction buffer (5×buffer=1 M potassium cacodylate, 0.125 M Tris-HCl, and 1.25 mg/ml bovine serum albumin at pH 6.6), 10 μl of 25 mM CoCl₂, 2.5 μl of a mixture of dATP (8 mM) and labeled dUTP (2 mM) and 5 μl of 25 units/μl terminal transferase.

The reaction mixture was incubated at 37° C. for 30 minutes and then placed on ice. In order to stop the reaction, 5 μL of a solution of glycogen in 0.2 M EDTA pH 8.0 (0.1 mg/mL) was added. The labeled DNA was purified by gel filtration chromatography (Centri-Sep, columns, Princeton Separations, Inc., Adelphia, N.J.). The labeled target DNA was detected by the method of Example 3.

### EXAMPLE 11

Labeling Target DNA by Random Primed-Labeling

Two μl of target DNA (preferably linearized) in 20 μl of Tris buffer or distilled water were denatured in a boiling water bath for two minutes and immediately placed on ice. After brief centrifugation at 4° C., the following compounds were added on ice: 35 μl of distilled water, 20 μl of 5×reaction buffer (1 M HEPES (pH 6.6), 250 mM Tris-HCl (pH 8.0), 25 mM MgCl₂, 100 mM NaCl and 10 mM dithiothreitol), 10 μl of 5′-sensitizer-labeled random hexamers at 620 OD/ml (final concentration of 62 OD/ml), 10 μl of mix of 1 mM each of: dCTP, dATP, dGTP, dTTP (0.1 mM final concentration of each), 5 μl of Klenow fragment at 2 units/μl for final 10 units/reaction.

The reaction mixture was incubated overnight at 37° C., followed by the addition of 10 μl of a 0.2 M EDTA solution (pH 8.0) to stop the reaction. In order to remove unincorporated nucleotides, 50 μl of the reaction mixture was passed through a Centri-Sep column (Princeton Separations, Incorporated, Adelphia, N.J.) according to directions of the manufacturer. The random primed-labeled target DNA was thereafter hybridized to the immobilized specific probe sequences of Example 12.

### EXAMPLE 12

Immobilization of Specific Oligonucleotide Probes on Aminofunctionalized Surfaces

The following immobilization of oligonucleotides was based on the formation of stable amide linkages between aminoalkyl modified polypropylene and carboxyfunctionalized oligonucleotides. For a single spot, 0.5 μL of a 20 μM activated oligonucleotide solution was used. 5′-carboxyfunctionalized oligonucleotides were activated with TSTU (O-(N -Succinimidyl)-1,1,3,3-tetramethyluronium-tetrafluoroborat) as follows:

Activation: 200 pmol carboxyfunctionalized oligonucleotide dissolved in 1 μL water were diluted with 5 μL DMSO. To this solution was added 2 μL of 0.3 mM diisopropylamine in DMSO and 2 μL of 0.3 mM TSTU in DMSO. The microtube was vortexed and centrifuged briefly. Then, the reaction mixtures were shaken at room temperature for 30 minutes.

Immobilization: 0.5 μL of the activation mixture of the carboxyfunctionalized oligonucleotide was spotted per dot on the surface of the aminoalkyl-membrane. After spotting, the membrane was incubated in a closed petri-dish at room temperature for 15 min and then washed as follows:

2×SSPE with 50% formamide, pH 7.4, at 65° C. for 1 h

DMF, at room temperature for 2 minutes, two times

DMF/H₂O (1:1, V:V), at room temperature for 2 minutes, two times

H₂O, at room temperature for 2 minutes, two times

absolute ethanol, at room temperature for 2 minutes, two times

The membrane was then dried at 45° C. during 30 minutes.

### EXAMPLE 13

Reverse Hybridization

Hybridization between the random-primed labeled target DNA prepared as described in Example 11 and the immobilized oligonucleotide probes described in Example 12 was performed as will now be described. Hybridization was carried out without blocking reagents, such as Denhardt's solution or heterologous DNA. Prehybridization was performed for 30 min at 45° C. in a buffer containing 0.25 sodium phosphate, pH 7.2, 7% SDS. After addition of the photosensitizer-labelled DNA fragment (10 pmol/cm²) (Example 11) the hybridization was run overnight at 45° C. in the dark. The membrane was hybridized under constant rotation in a thermoregulated hybridization oven. The buffer volumes was 1 ml/cm² membrane. After hybridization, the membrane was washed with the following buffers: 15 min in 6×SSC at room temperature, 5 min in 3×SSC at 45° C., 5 min in 3×SSC and twice for 5 min in hybridization buffer at room temperature. The membrane was then allowed to dry at room temperature.

### EXAMPLE 14

Detection of the Labeled Analyte After Hybridization

This example describes the method used to the detect hybridized DNA in Example 13.

The membrane was dipped in a solution of olefin (2-[3-(hydroxyphenyl) -methoxymethylene] adamantane 10 mg/100 ml n-hexane). After drying for 3 minutes in an hybridization oven at 40° C., the membrane was placed with its spots side on a red filter letting 670 nm light through it. A glass plate and a black non-reflecting background were placed on the membrane. It was illuminated with a 600W tungsten light source for 15 min. Then in a dark room, the membrane was placed on a heated aluminum plate, that was fitted to the opening of a Polaroid Land Pack Film Holder #405. The Holder with a Polaroid Film 667 (3000 ISO sensitivity) was placed on the aluminum plate with the membrane during 15 min. The film was developed (20 sec) and the signals analyzed. Alternatively, the signal on the heated membrane may be recorded with a CCD-camera.

### EXAMPLE 15

Incorporation of a Sensitizer-Labeled UTP in Target RNA by T7 Polymerase

The objective is to conduct a standard transcriptional runoff with T7 polymerase using a labeled ribonucleotide.

The template chosen for transcription was pCCSX. It consists of a ˜1.5 kb fragment of DNA (amplified by PCR) and cloned into the Sma 1 site of pGEM3Z-(Promega, Madison, Wis.). pCCSX was linearized by digestion with BAM H1 (New England Biolabs, Beverly Mass.) as recommended by the Bam H1 manufacturer. All Nucleotide TriPhosphates (NTPs) were purchased from Gibco Life Technologies (Gaithersburg, Md.) and diluted to 10 mM concentrations; T7 Polymerase, as well as the corresponding 10x Transcription buffer, was purchased from New England Biolabs (Beverly, Mass.). All other chemicals were purchased from Sigma Chemicals (St. Louis, Mo.). RNAse free DNAse 1 was obtained from Sigma Chemicals (St. Louis, Mo.). Methylene Blue labeled UTP (UTP^{mb}) was synthesized as described in Example 4.

Reactions were set up as follows: 1.0 μl; of UTP/UTP^{mb} A or B, 1.0 μl each of 10 mM ATP, 10 mM GTP and 10 mM CTP, 2.0 μl of 10×Buffer 10×buffer=(200 mM Tris-HCl (pH 8.0), 40 mM MgCl₂, 10 mM spermidine, (250 mM NaCl), 500 ng template (linear), 5 units T₇ polymerase, q.s. to 20 μl with dd water. Component UTP/UTP^{mb} A of the reaction mixture was prepared as follows: 2.5 μl of a ⅕ dilution of UTP^{mb} stock solution at approximate 50 mM concentration was mixed with 7.5 μl UTP (10 mM). Component UTP/UTP^{mb} B of the reaction mixture was prepared as follows: 2.5 μl of an undiluted UTP^{mb} stock solution at about 50 mM was mixed with 7.5 μl UTP (10 mM). The reaction was incubated for 2 hours at 37° C., stopped by the addition of 2 μL 0.2 M EDTA (pH 8.0) and mixed by vortexing.

Reactions were set up three times for each set. Control reactions consisted of no MB labeled UTP, and UTP^{mb} with no template. Each reaction was cleaned to remove unincorporated UTP^{mb} using a Centri-Sep column as described in previous examples and detected as described in Example 3.

### EXAMPLE 16

Detection of Creatine Kinase in Antibody Serum

Antibody specific for creatine kinase M subunit was purified from CK-MB reagent (Sigma) and spotted onto a nitrocellulose membrane. One hundred microliters of suspected Human Creatine Kinase (CK) isoenzyme from a purification preparation was labeled with a succinimidyl ester of Methylene Blue using a Methylene Blue Protein Labeling Kit in accordance with the directions of the kit manufacturer (EMP Biotech, Berlin, Germany). The membrane was contacted with the labeled material for 60 minutes at 37° C. in PBS, washed extensively to remove unreacted material and dried. The membrane was coated with an olefin solution, dried and irradiated with light at 670 nm for 15 minutes. The membrane was then flooded with 0.1N NaOH, drained, wrapped in plastic and contacted with autoradiography film for 1 hour. Upon development, the film showed the presence of dark spots over the area where the antibody had been spotted.

What is claimed is:

1. A method for detecting an analyte in a sample comprising the steps of:
(a) exciting a sensitizer label on an analyte;
(b) permitting energy from the excited sensitizer label to be transferred to and excite an acceptor molecule, whereby the sensitizer label returns to an unexcited state;
(c) reacting the excited acceptor molecule with a chemiluminescent precursor to form a chemiluminescent compound which emits light in response to an activation source;
(d) exposing the chemiluminescent compound to the activating source to produce a detectable signal;
(e) detecting said signal; and
(f) correlating the signal with the presence or absence of the analyte.

2. The method of claim 1, further comprising the step of measuring the amount of signal produced, wherein the amount of the signal is correlated to the amount of analyte present in the sample.

3. The method of claim 1, further comprising the step of immobilizing the labeled analyte on a carrier.

4. The method of claim 3, wherein the carrier is selected from the group consisting of membrane, glass, gel, emulsion, film, and combinations thereof.

5. The method of claim 1, wherein the analyte is selected from the group consisting of polynucleotide, protein, peptide, polypeptide, saccharide, polysaccharide, peptide nucleic acid, antigen, hapten, antibody, and combinations thereof.

6. The method of claim 7, wherein the analyte is a polynucleotide selected from the components of DNA, RNA or a fragment thereof.

7. The method of claim 6, wherein the polynucleotide analyte is labeled by incorporation of a sensitizer-labeled nucleotide during a nucleic acid amplification reaction, primer extension reaction, or in vitro transcription reaction

8. The method of claim 6, wherein the polynucleotide analyte is labeled using sensitizer-labeled primers during a nucleic acid amplification reaction, primer extension reaction, or in vitro transcription reaction.

9. The method of claim 7, wherein the amplification reaction is selected from the group consisting of PCR, RT-PCR, NASBA, LCR, SAGE, and differential display.

10. The method of claim 8, wherein the amplification reaction is selected from the group consisting of PCR, RT-PCR, NASBA, LCR, SAGE, and differential display.

11. The method of claim 8, wherein the primers are random primers.

12. The method of claim 8, wherein the primers are specific primers.

13. The method of claim 5, wherein the polynucleotide analyte is hybridized to mutation-specific nucleic acid sequences bound to a carrier.

14. The method of claim 1, wherein the sensitizer is exposed to light having a wavelength from about 30 nm to about 1,100 nm to excite the sensitizer.

15. The method of claim 1, wherein said signal is detected optically.

16. The method of claim 1, wherein the signal is light energy.

17. The method of claim 16, wherein the light energy is detected by light-sensitive film.

18. The method of claim 16, wherein the light energy is detected by a photoelectric cell.

19. The method of claim 1, wherein the acceptor molecule is molecular oxygen in the ground state.

20. The method of claim 1, wherein the chemiluminescent precursor is an olefin selected from the group consisting of enol ethers, enamines, 9-alkylidene-N-alkylacridans, arylvinylethers, 1,4-dioxenes, 1,4-thioxenes, 1,4-oxazines, arylimidazoles, 9-alkylidene-xanthenes and lucigenin.

21. The method of claim 1, wherein the sensitizer is a dye.

22. The method of claim 21, wherein the dye is selected from the group consisting of methylene blue, porphyrins, metalloporphyrins, aromatic hydrocarbons, pyrenes, phthalocyanine, hemin, flavin derivatives, xanthines, tri-aryl methanes, phenothiazines, and rhodamine heterocyclic compounds.

23. The method of claim 1, wherein the chemiluminescent precursor is in a dry state on a carrier.

24. The method of claim 1, wherein the activating source is a chemical base and/or heat.

25. The method of claim 1, wherein the chemiluminescent compound is a dioxetane that decomposes upon exposure to the compound activation source to produce the detectable signal.

26. The method of claim 1, wherein the activating source is incorporated into a carrier.

27. A method for detecting an analyte in a sample comprising the steps of:
(a) immobilizing a sensitizer-labeled analyte on a carrier;
(b) exposing the immobilized analyte to light of an appropriate wavelength to electronically excite the sensitizer;
(c) permitting energy from the excited sensitizer label to be transferred to and excite an acceptor molecule, whereby the sensitizer label returns to an unexcited state;
(d) reacting the excited acceptor molecule with a chemiluminescent precursor to form a chemiluminescent compound which emits light in response to an activation source;
(e) exposing the chemiluminescent compound to the activating source to produce a detectable signal;
(f) detecting the signal; and
(g) correlating the signal with the presence or absence of the analyte in the sample.

28. The method of claim 27, further comprising the step of measuring the amount of signal produced, wherein the amount of the signal is correlated to the amount of analyte present in the sample.

29. The method of claim 27, wherein the analyte is a nucleic acid.

30. The method of claim 29, wherein the analyte is labeled by incorporation of a sensitizer-labeled nucleotide during a target amplification reaction, primer extension reaction, or in vitro transcription reaction.

31. The method of claim 29, wherein the analyte is labeled using sensitizer-labeled amplification primers during a target amplification reaction, primer extension reaction, or in vitro transcription reaction.

32. The method of claim 29, wherein the analyte is DNA, RNA, peptide nucleic acid or a fragment thereof.

33. The method of claim 27, wherein the sensitizer is exposed to light having a wavelength of about 30 nm to about 1,100 nm to excite the sensitizer.

34. The method of claim 27, wherein the light energy is detected by light-sensitive film.

35. The method of claim 27, wherein the light energy is detected by a photoelectric cell.

36. The method of claim 27, wherein the chemiluminescent precursor is in a solid state on a carrier.

37. The method of claim 27, wherein the activating source is incorporated into a carrier.

38. The method of claim 27, wherein the carrier is selected from the group consisting of membrane, glass, gel, emulsion, film, and combinations thereof.

39. A method for detecting a specific nucleotide sequence in a polynucleotide analyte comprising the steps of:
(a) providing a sensitizer-labeled analyte;
(b) providing the specific sequence on a carrier;
(c) hybridizing the labeled analyte to the specific sequence, thereby forming a hybridization complex;
(d) exposing the hybridization complex to light of an appropriate wavelength to electronically excite the sensitizer;
(e) permitting energy from the excited sensitizer label to be transferred to and excite an acceptor molecule, whereby the sensitizer label returns to an unexcited state;
(f) reacting the excited acceptor molecule with a chemiluminescent precursor to form a chemiluminescent compound which emits light in response to an activation source;
(g) exposing the chemiluminescent compound to the activating source to produce a detectable signal;
(h) detecting the signal; and
(i) correlating the signal with the presence or absence of the analyte in the sample.

40. The method of claim 39, further comprising the step of measuring the amount of signal produced, wherein the amount of the signal is correlated to the amount of polynucleotide analyte present in the sample.

41. The method of claim 39, wherein the analyte is labeled by incorporation of a sensitizer-labeled nucleotide during a target amplification reaction, primer extension reaction, or in vitro transcription reaction.

42. The method of claim 39, wherein the analyte is labeled using sensitizer-labeled primers during a target amplification reaction, primer extension reaction or in vitro transcription reaction.

43. The method of claim 40, wherein the primers are random primers.

44. The method of claim 40 wherein the primers are specific primers.

45. The method of claim 39, wherein the specific sequence to be detected in the analyte is a mutation-specific sequence.

46. The method of claim 39, wherein the analyte is DNA, RNA, peptide nucleic acid or a fragment thereof.

47. The method of claim 39, wherein the sensitizer is exposed to light having a wavelength from about 30 nm to about 1,100 nm to excite the sensitizer.

48. The method of claim 39, wherein the light energy is detected by light-sensitive film.

49. The method of claim 39, wherein the light energy is detected by a photoelectric cell.

50. The method of claim 39, wherein the chemiluminescent precursor is in a solid state on a carrier.

51. The method of claim 39, wherein the activating source is incorporated into a carrier.

52. A method of determining if a patient is at risk for a disorder or has a disorder comprising detecting in a patient specimen the presence or absence of a lesion of an analyte, wherein said detecting comprises:
(a) providing a sensitizer-labeled analyte;
(b) exciting the sensitizer;
(c) permitting energy from the excited sensitizer label to be transferred to and excite an acceptor molecule, whereby the sensitizer label returns to an unexcited state;
(d) reacting the excited acceptor molecule with a chemiluminescent precursor to form a chemiluminescent compound which emits light in response to an activation source;
(e) exposing the chemiluminescent compound to the activating source to produce a detectable signal;
(f) detecting said signal and/or the amount of the signal; and
(g) correlating the signal and/or amount of the signal with the presence or absence of the lesion of the analyte in the patient specimen as compared to a control patient specimen.

53. The method of claim 52, wherein said lesion is characterized by an alteration in sequence, expression, post-translation modification, or some combination thereof of the analyte.

54. The method of claim 52, wherein the analyte is selected from the group consisting of polynucleotide, protein, peptide, polypeptide, saccharide, polysaccharide, hapten, peptide nucleic acid, antigen, antibody and combinations thereof.

55. The method of claim 54, wherein the polynucleotide is DNA, RNA or a fragment thereof.

56. The method of claim 55, wherein the polynucleotide analyte is labeled by incorporation of a sensitizer-labeled nucleotide during a target amplification reaction, primer extension reaction, or in vitro transcription reaction.

57. The method of claim 55, wherein the polynucleotide analyte is labeled using sensitizer-labeled primers during a target amplification reaction, primer extension reaction, or in vitro transcription reaction.

58. The method of claim 55, wherein the labeled analyte is hybridized to a carrier containing an array of oligonucleotides representing potential mutations in the analyte.

59. A system for detecting an analyte comprising:
(a) an analyte labeled with a sensitizer moiety;
(b) a chemiluminescent precursor compound capable of forming a chemiluminescent compound which emits light in response to an activation source; and
(c) activating source capable of causing the chemiluminescent compound to produce a detectable signal.

60. The system of claim 59 further including a means for detecting the signal.

61. The system of claim 59 wherein the chemiluminescent precursor is an olefin capable of reacting with singlet oxygen and converting to a metastable dioxetane.

62. The system of claim 59, wherein the analyte is bound to a carrier.

63. The system of claim 62, wherein the chemiluminescent precursor is present in a dry state on a second carrier.

64. The system of claim 63, wherein the activating source is incorporated into a third carrier.

65. The system of claim 59, wherein the chemiluminescent precursor is present in a dry state on a carrier.

66. The system of claim 59, wherein the activating source is incorporated into a carrier.

67. The system of claim 59, wherein the signal is detected by light-sensitive film.

68. The method of claim 59, wherein the signal is detected by a photoelectric cell.

69. A kit for detecting an analyte comprising:
(a) an analyte labeled with a sensitizer moiety; and
(b) a chemiluminescent precursor compound capable of forming a chemiluminescent compound which emits light in response to an activation source.

70. The kit of claim 69 further including an activating source capable of causing the chemiluminescent compound to produce a detectable signal.

71. The kit of claim 69 further including a means for detecting the signal.
